(19) United States
(12) Patent Application Publication
(10) Pub. No.: US 2010/0106012 A1
(43) Pub. Date: Apr. 29, 2010

(54) PRESSURE-RESISTANT 3-WAY STOPCOCK

(75) Inventor: Emiliano De Marco, Loncin (BE)

Correspondence Address: GE HEALTHCARE, INC., IP DEPARTMENT 101 CARNEGIE CENTER, PRINCETON, NJ, 08540-6231, US

(73) Assignee: GE MEDICAL SYSTEMS BENELUX S.A., Loncin (BE)

(21) Appl. No.: 12/442,578

(22) PCT Filed: Sep. 28, 2007
(86) PCT No.: PCT/EP07/08472

§ 371 Date: Nov. 23, 2009

Related U.S. Application Data
(60) Provisional application No. 60/848,339, filed on Sep. 29, 2006.

(51) Int. Cl. A61M 39/22; F16K 11/085

(52) U.S. Cl. 600/431; 137/625.47

(57) ABSTRACT

A pressure-resistant and chemical-resistant stopcock valve (10) includes an elongate cylindrical wall (14) and a bracing wall (28) spanning across the internal cavity. The cylindrical wall and the bracing wall define a through passage (32) extending across the stopcock body. The bracing wall provides additional support to the stopcock about the flowports at either end of the passage to better maintain fluid integrity with the associated manifold into which the stopcock is inserted.

### FIELD OF THE INVENTION

The present invention is directed to the field of pharmaceutical handling equipment. More specifically, the present invention is directed to a stopcock for a stopcock valve.

### BACKGROUND OF THE INVENTION

The art has seen synthesizers for producing radioactive tracers for human injection as used for Positron Emission Tomography (PET), i.e. medical diagnosis through nuclear imaging. The synthesizer use a consumable component, also called a cassette, which mounts to a control unit. The cassette includes all of the chemicals involved in the manufacture of the tracer while the control unit operates the cassette through the opening and closing of valves thereon so as to direct the constituent components through a manufacturing process which produces the tracer as an output. The chemicals within the cassette only come into contact with a disposable fluid path (i.e. the cassette). After each run, the cassette may be replaced with a new cassette to begin a new tracer production cycle. For cassette manufactured by the assignee of the present invention as FastLab™, the basic skeleton of the cassette consists of a manifold of twenty-five 3-way stopcock valves which are sequentially set to provide different flow-paths in fluid communication with the manufacturing order. As the cassette is a consumable is made of an inexpensive plastic material.

The stopcock valves for the cassette must be inexpensive, chemically resistant (towards organics solvents, acids and alkaline solutions), suitable for pharmaceutical applications, resistant to temperatures above 100° C., and resistant to a pressure of 5 bars. The cost criteria and the requirement for a chemically-resistant material suitable for pharmaceutical applications led to a very limited choice of plastic materials. Basically only polypropylene satisfied all these criteria. Polypropylene is a soft material which creeps when submitted to a constraint, such as that experienced by a stopcock valve inserted into the barrel, or stopcock housing, of the manifold.

When the stopcocks are assembled into the barrels, the constraint of the barrels about the stopcock leads to a creep effect, i.e. the dimension and shape of the stopcock changes under the constraint. The result of this creep effect is that the stopcock valves are no longer leak-tight at 5 bars after an accelerated aging simulation by placing the valves into a thermostatic cell at 60° C. to simulate 2 year aging within 2 month, even though the valves were leak-tight before the aging simulation.

There are commercially available 3 way-stopcock manifolds. Most commercially available 3 way-stopcock valves are made of one hard material (for example polysulfone or polycarbonate) for the valve housing and one soft material for the stopcock itself (for example Polyethylene or Polypropylene). The use of a hard material for the stopcock housing prevents creep and dimensional/shape changes under constraint. These valves are resistant to pressure above 5 bars but the material (polysulfone or polycarbonate) is not chemically resistant. There are also few commercial stopcock valves made of polypropylene or polyethylene (soft material) for housing and stopcock. These valves are chemically resistant but do not resist to pressure. According to supplier the pressure resistance is limited to 1-2 bars.

Commercial polysulfone manifolds with polyethylene valves are used on the cassette for the synthesizer marketed by the assignee of the present invention under the tradename TRACERLab MX. Polysulfone, however, is not suitable for FastLab™ as it is not resistant to some of solvents used therein (e.g. DMSO).

There is therefore a need in the art for a stopcock valve that is both chemically-resistant as well as pressure resistant. Desirably, there is a need for a stopcock valve that is chemically-resistant and able to withstand a pressure of 5 bars.

A cassette for use with the present invention is disclosed in EP 836 609, the entire contents of which are hereby incorporated by reference. Another suitable cassette is shown in FIG. 11.

The low dead volume stopcock valves were disclosed in commonly assigned

United States Patent Publication No. US2005139276, the entire contents of which are incorporated herein by reference.

### SUMMARY OF THE INVENTION

In view of the needs of the prior art, the present invention provides a stopcock for a stopcock valve. The stopcock includes a stopcock body having an elongate cylindrical wall having a cylindrical exterior surface and a cylindrical interior surface. The cylindrical wall defines a first open end and first and second opposed valve ports. The interior surface defines an elongate first cylindrical stopcock cavity in fluid communication with the open end. A bracing wall spans the stopcock cavity and includes at least a first major surface facing the stopcock cavity. The bracing wall defines an open elongate through-passage extending across the stopcock body in fluid communication with said first and second valve ports.

### BRIEF DESCRIPTION OF THE DRAWINGS

FIG. 1 depicts the stopcock of the present invention.

FIG. 2 depicts an oblique view of the stopcock of FIG. 1.

FIG. 3 depicts a cross-sectional view of the stopcock of FIG. 1, taken through the line 3-3.

FIG. 4 depicts an alternate embodiment of stopcock of the present invention.

FIG. 5 depicts yet another embodiment of stopcock of the present invention.

FIG. 6 depicts still another embodiment of stopcock of the present invention.

FIG. 7 depicts a top elevational view of the stopcock of FIG. 1.

FIG. 8 is an alternate depiction of the stopcock of the present invention.

FIG. 9 is a cross-sectional view of the stopcock of FIG. 7, taken through the lines 9-9.

FIG. 10 depicts a pair of stopcocks of the present invention, positioned within their respective manifolds, during a test to demonstrate fluid integrity.

FIG. 11 depicts a cassette having manifolds for receiving stopcocks of the present invention.

FIGS. 12A-F depict the operation of a stopcock of the present invention within a manifold.

### DETAILED DESCRIPTION OF THE PREFERRED EMBODIMENT

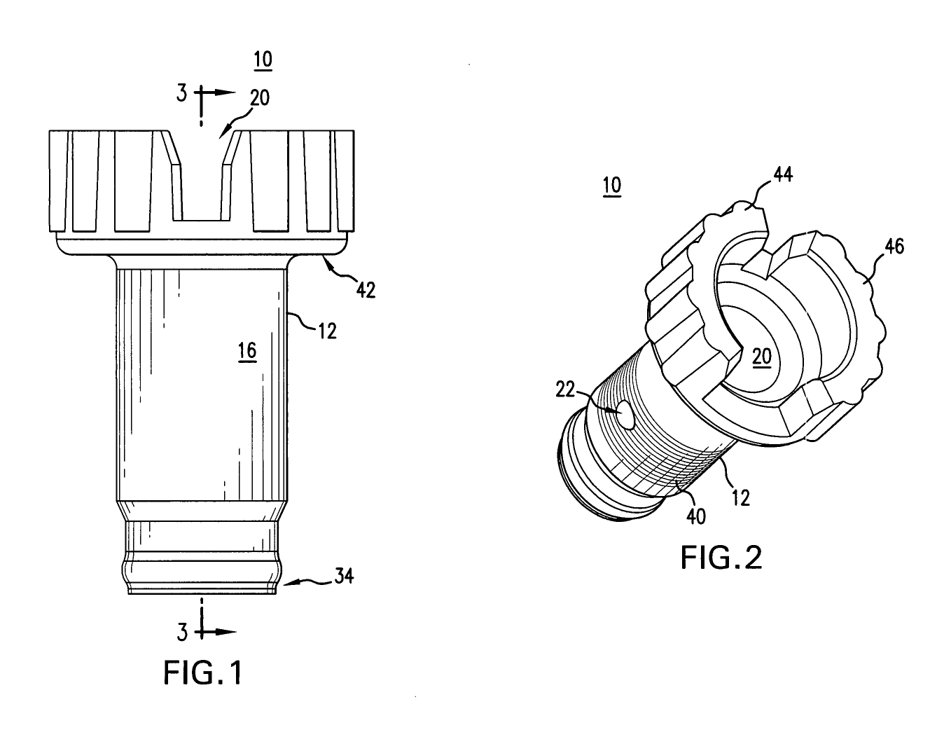

FIG. 1 depicts a stopcock 10 of the present invention. Stopcock 10 is desirably formed from relatively soft plastic material, such as polypropylene, which exhibit sufficient chemical resistance to the chemicals with which it will come into contact during operation. Manufacture of stopcock 10 is desirably performed by injection molding. It is further contemplated that stopcock 10 may alternatively be formed from polyethylene and any other relatively soft materials which exhibit the desired chemical resistance and sufficient temperature resistance to deformation which might be experienced during operation. Polyethylene, for example was not chosen for the FastLab™ application due to its lower temperature resistance.

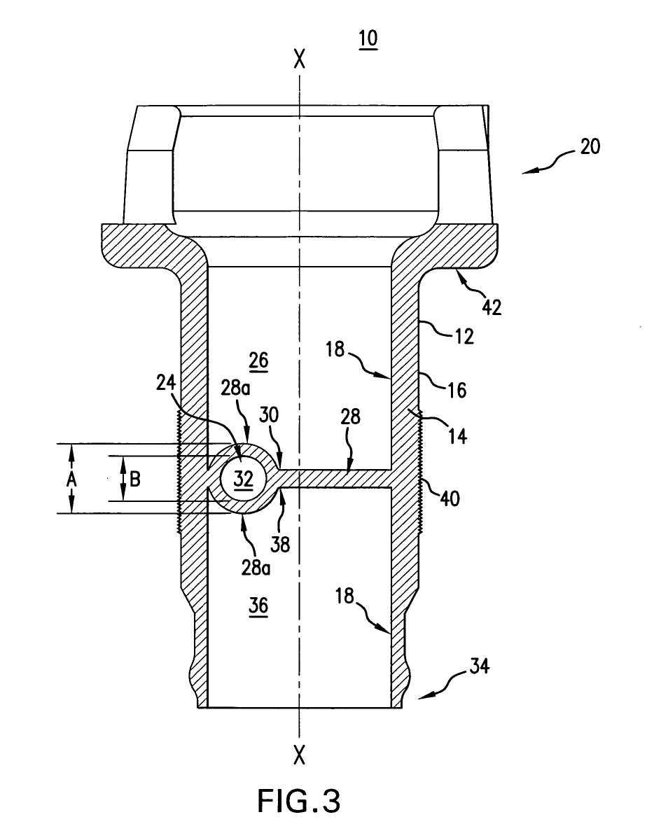

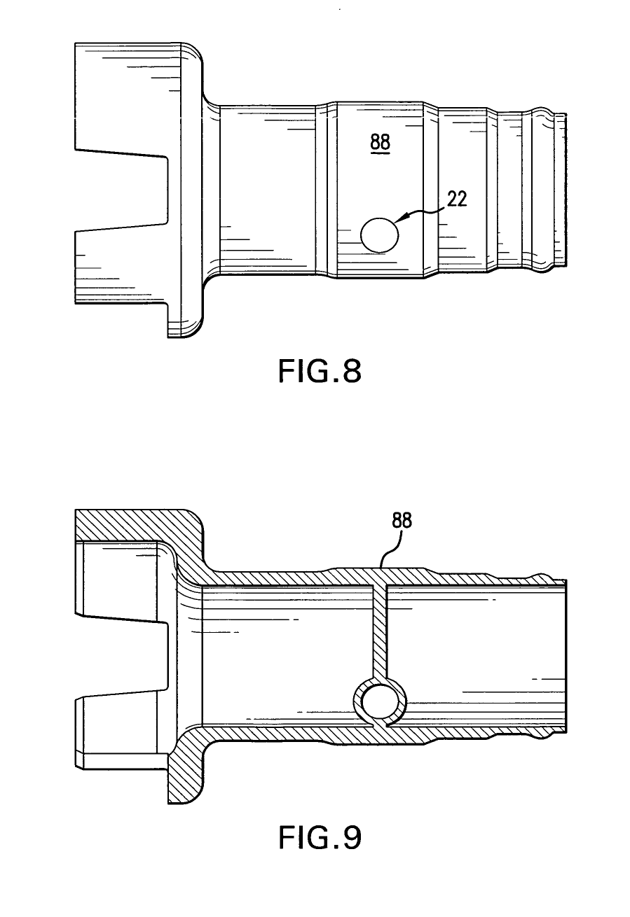

With additional reference to FIGS. 2, 3, 8 and 9, stopcock 10 includes a stopcock body 12 having an elongate cylindrical wall 14. Cylindrical wall 14 includes a cylindrical exterior surface 16 and a cylindrical interior surface 18. Cylindrical wall 14 also defines a first open end 20 and first and second opposed valve ports 22 and 24, respectively. Interior surface 18 defines an elongate first cylindrical stopcock cavity 26 in fluid communication with open end 20. Bracing wall 28 spans stopcock cavity 26 and includes first major surface 30 facing first stopcock cavity 26. Bracing wall 28 defines open elongate through-passage (or channel) 32 extending therethrough in fluid communication with first and second valve ports 22 and 24. Channel 32 desirably extends transversely along an off-center cord-line, so as not to intersect with the center axis X, through stopcock body 12 so as to minimize the volume of material which will be held therein at any given time. This off-center cord-line is alternatively described as extending adiametrically across cylindrical wall 14. While in the embodiment of FIGS. 1-3, valve ports 22 and 24 are radially-spaced 120 degrees about the elongate center axis X of stopcock 10, the present invention further contemplates that other non-diametrical spacing of the valve ports may be employed.

As bracing wall 28 and passage 32 is desirably centrally-located along the length of stopcock body 12, cylindrical wall 14 desirably defines a second open end 34 and interior surface 18 defines a second elongate cylindrical stopcock cavity 36 in fluid communication therewith. Bracing wall 28 includes a second major surface 38 facing second stopcock cavity 36. Bracing wall 28 provides a counterbalance to the external constraint from the manifold barrel into which it is inserted and thereby prevents changes in the shape or dimensions of stopcock 10 which result in leaking between the stopcock and manifold during operation.

First and second planar surfaces 30 and 38 extend transversely across first and second stopcock cavities 26 and 36, respectively, so as to be in facing opposition to first and second open ends 20 and 34, respectively of cylindrical wall 14. Through-passage 32 extends along a chord line of cylindrical wall 14. The chord line of passage 32 desirably does not cross the geometric center of the cross-section of cylindrical wall 14, that is, it does not intersect the rotational axis X. It is further contemplated that bracing wall 28 further comprises an arcuate portion 28a extending about through-passage 32. First and second planar surfaces 30 and 38 define a wall thickness therebetween that is smaller in dimension than the maximum transverse span A of arcuate portion 28a. Desirably, first and second planar surfaces 30 and 38 define a wall thickness therebetween that is smaller in dimension than the maximum transverse span B of through-passage 32.

It is further contemplated that cylindrical wall 14 includes a plurality of elongate micro-grooves 40 along exterior surface 16 to either side of said first and second ports 22 and 24. As stopcock body 12 is desirably formed by injection molding, it is contemplated by the present invention that micro-grooves 40 may be formed either by providing the negatives of grooves 40 on the mold tools or by machining micro-grooves 40 onto surface 16 after stopcock body has been removed from the mold. It is further contemplated that the provision of microgrooves 40 onto a stopcock body that has been injection molded is a separate invention of its own right.

Stopcock 10 includes an annular radial flange 42 transversely extending from first open end 20 of cylindrical wall 14. Flange 42 supports a pair of manually-engageable upstanding perimetrical tabs 44 and 46 extending substantially normally thereto. Desirably, tabs 44 and 46 are non-symetrically spaced about the longitudinal axis of the stopcock body so as to provide non-uniform gaps therebetween. The gaps are contemplated to be matingly engaged with an automated stopcock turning mechanism which fills in each of the different-size gaps (ie, keyed to the tabs) so that the stopcock turning mechanism will have predetermined confirmation of the orientation of the stopcock prior to rotating the stopcock within its manifold.

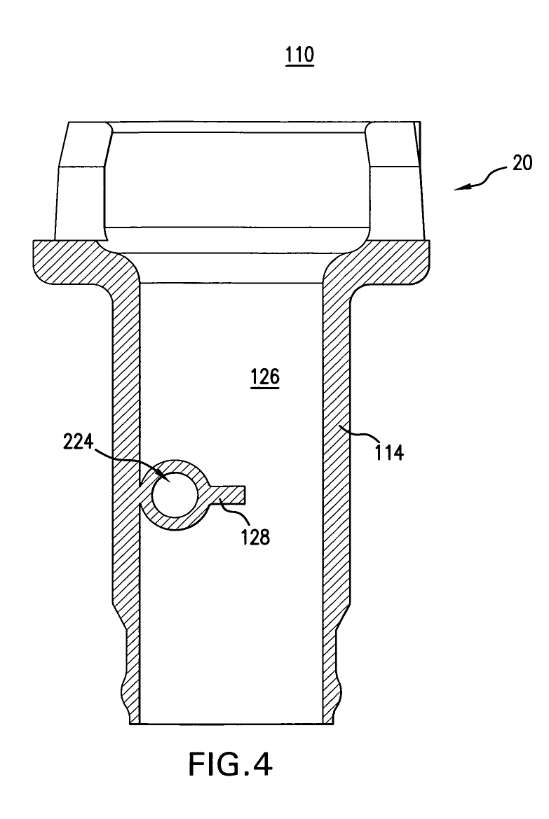

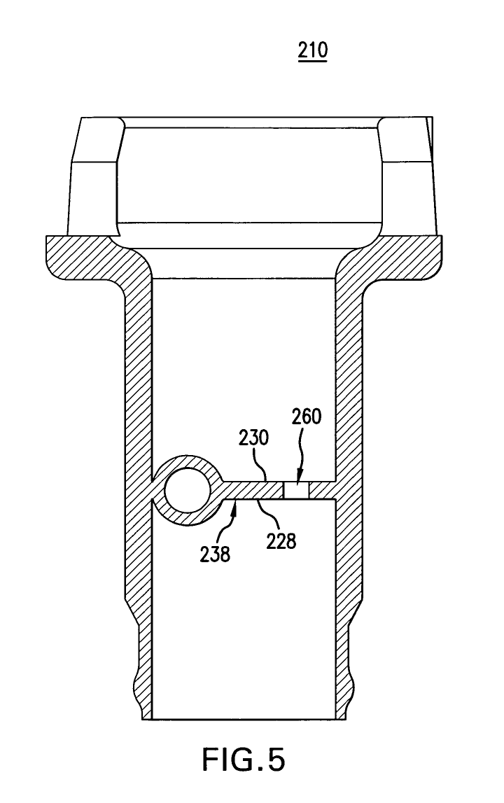

It is further contemplated that bracing wall need not extend fully across so as to fluidly isolate the first and second cavities from each other. As shown in FIG. 4, the present invention further contemplates a stopcock 110 having a bracing wall 128 which only spans partway transversely across the first and second cavities 126 and 226 so as to still provide a region of increased rigidity to cylindrical wall 114 about each of ports 222 and 224. Desirably, bracing wall 128 extends across cavity 126 beyond the center point of the cross-section of cylinder 14. Alternatively still, as shown in FIG. 5, the present invention contemplates a stopcock 210 having a bracing wall 228 which fully spans across first and second cavities 226 and 236 defines an aperture 260 extending between major surfaces 230 and 238 in fluid communication with cavities 226 and 236.

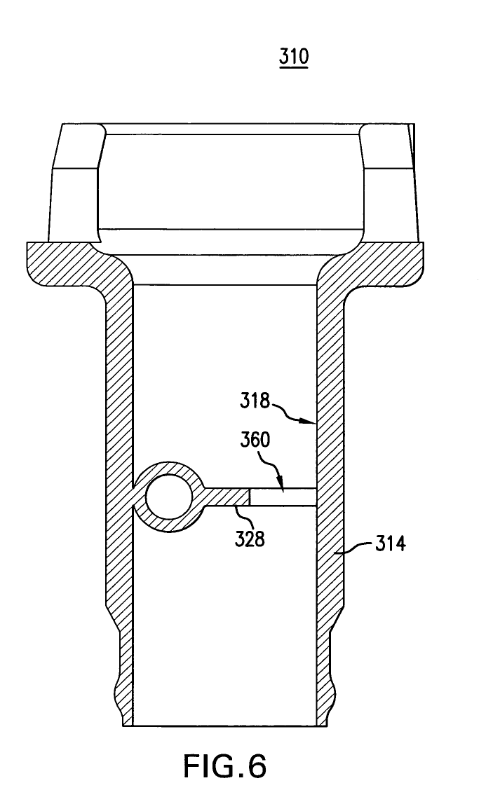

With reference to FIG. 6, yet another stopcock 310 of the present invention includes a bracing wall 328 defining an aperture 360 therethrough which opens on a portion of inner surface 318 of cylindrical wall 314. It is further contemplated that the bracing wall of the present invention need not be of uniform thickness, it may, for example provide a cross-sectional shape with upstanding flanges so as to impart a higher bending inertia to the bracing wall for better supporting the ports against the barrel of the manifold.

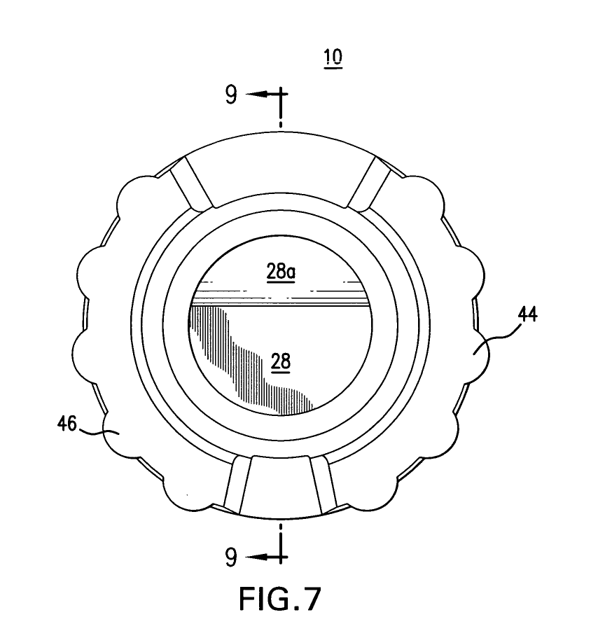

FIGS. 7-9 depict another stopcock of the present invention which is contemplated to be the same as stopcock 10 except where noted. FIG. 9 is a cross-sectional view of the stopcock of FIG. 7, taken through the lines 9-9. The stopcock of FIGS. 7-9 includes region of increased diameter so as to provide a raised surface 88 extending beyond the cylindrical body of the stopcock and provide localized interaction between the stopcock and a manifold about channel 22. Raised surface 88 provides a smaller area of frictional fit with the manifold, yet maintains a sufficient interference fit therewith to maintain fluid integrity about channel 22 and prevent leakage between the stopcock and manifold.

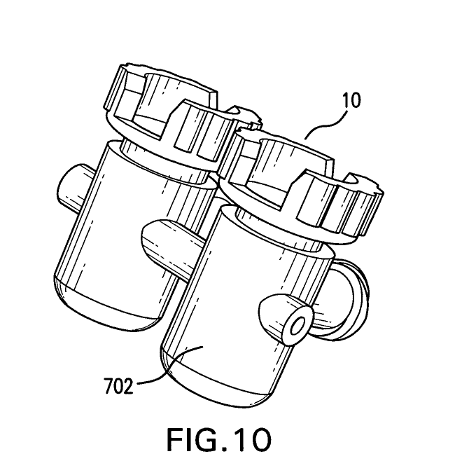

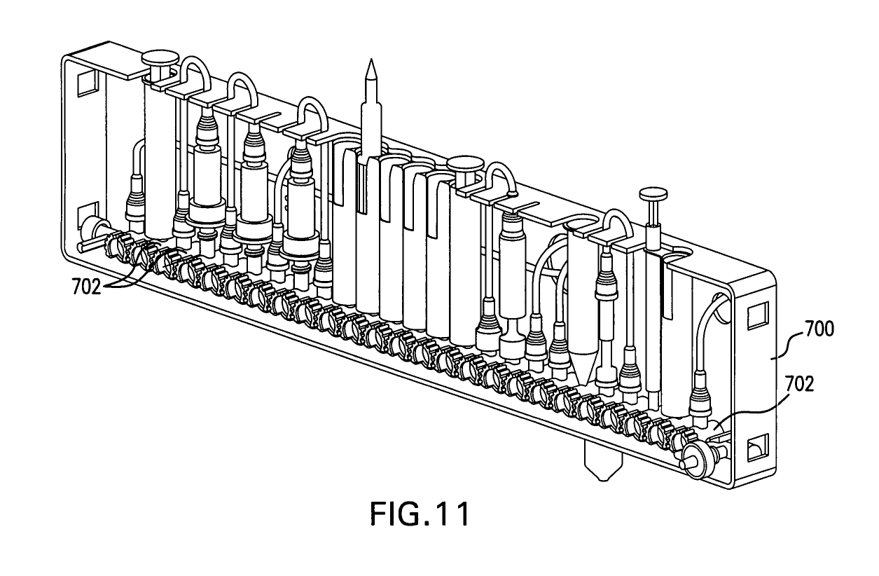

FIG. 11 depicts a cassette 700 having a number of manifolds 702 for receiving a stopcock of the present invention. Cassette 700 is desirably formed from the same material used to form the inserted stopcocks. FIG. 10 depicts a pair of stopcocks 10 within their manifold 702 isolated from cassette 700.

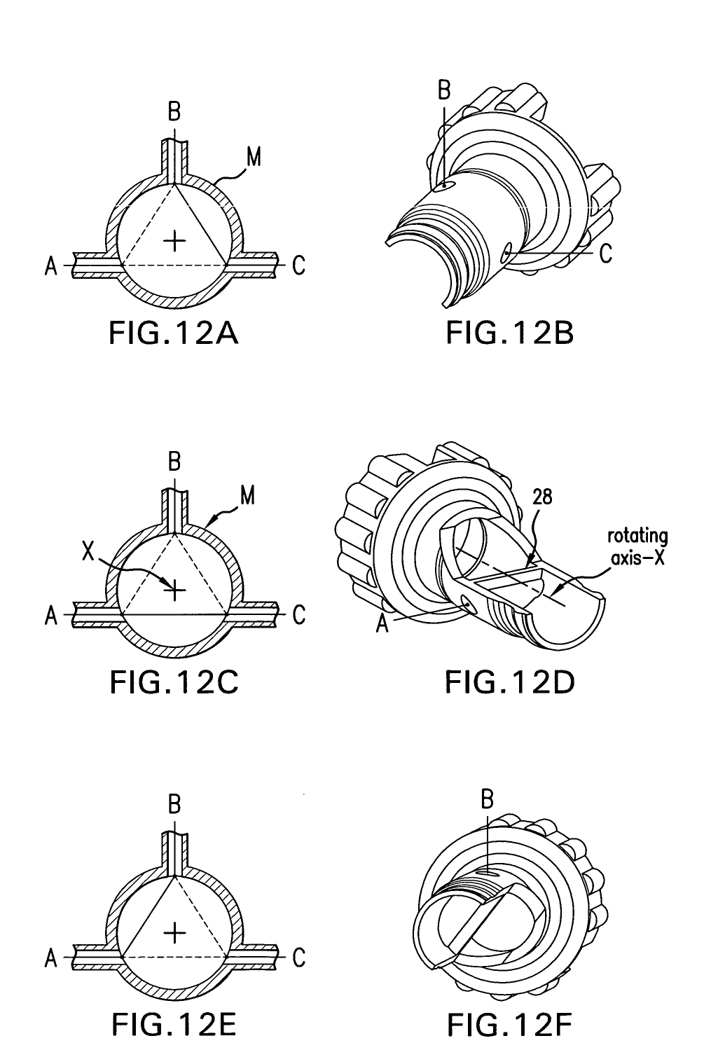

FIGS. 12A-F further depict a stopcock of the present invention in operation. As shown in FIG. 12A, a manifold M includes a cylindrical wall defining ports A, B, and C. A stopcock of the present invention is fit into manifold M and rotated so as to position fluid channel 32 to be extending in fluid communication between any two of ports A, B, and C of manifold M. FIGS. 12A, 12C, and 12E depict channel 32 extending in fluid communication between each ports B-C, A-C, A-B, respectively.

Each alignment between ports A, B, and C is achieved by rotating stopcock 10 about axis X. In FIGS. 12B, 12D, and 12F, the stopcock body is shown in a partial cut-away view so as to reveal bracing wall 28 and better present the orientation of the bracing wall's channel within the stopcock at each orientation.

The present invention therefore provides a stopcock valve design which is both pressure resistant and chemically resistant. The stopcocks of the present invention include a reinforing bracing wall behind the ports on the outer surface of the stopcock, ie, where leak-tightness is required (around the channel through the stopcock). The provision of a bracing wall counter-acts the external constraint from the barrel of the manifold into which it has been inserted and thus prevents changes in the shape and dimensions in the stopcock which result in leakage during operation.

A feasibility study performed on stopcock 10 yielded extremely good results as the stopcocks remained leak-tight at 8 bars after a 12 month accelerated aging simulation. The tested stopcocks, made of polypropylene, were assembled into the manifold of a cassette made of the same grade of polypropylene. These stopcocks exhibited leak-tightness at up to 8 bars after an accelerated aging process simulating 12 month. Without this reinforcement bracing wall of the present invention, leaks are observed already at 1 bar after accelerated aging simulating 6 month, with the same material for stopcock and cassette.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A stopcock for a stopcock valve, said stopcock comprising:
A stopcock body having an elongate cylindrical wall having a cylindrical exterior surface and a cylindrical interior surface, said cylindrical wall defining a first open end and first and second opposed valve ports, said interior surface defining an elongate first cylindrical stopcock cavity in fluid communication with said open end;
a bracing wall spanning said stopcock cavity, said bracing wall including at least a first major surface facing said stopcock cavity,
wherein said bracing wall defines an open elongate through-passage extending across said stopcock body in fluid communication with said first and second valve ports.

2. The stopcock of claim 1, wherein said cylindrical wall defines a second open end and said interior surface defines a second elongate cylindrical stopcock cavity in fluid communication therewith, said bracing wall including a second major surface in facing said second stopcock cavity.

3. The stopcock of claim 2, wherein said bracing wall includes a planar portion having opposed first and second planar surfaces.

4. The stopcock of claim 3, wherein said bracing wall further comprises an arcuate portion, said arcuate portion extending about said through-passage.

5. The stopcock of claim 2, wherein said first and second planar surfaces define a wall thickness therebetween that is smaller in dimension than the maximum transverse span of said through-passage.

6. The stopcock of claim 2, wherein said first and second planar surfaces of said bracing wall extend transversely across said first and second stopcock cavity so as to be in facing opposition to said first and second open ends of said cylindrical wall.

7. The stopcock of claim 1, wherein said through-passage extends adiametrically across said stopcock body.

8. The stopcock of claim 1, wherein said cylindrical wall defines a plurality of elongate micro-grooves along said exterior surface to either side of said first and second ports.

9. The stopcock of claim 1, wherein said exterior surface is contoured to define a region of increased diameter along the length thereof which defines said first and second valve ports.

10. The stopcock of claim 1, further comprising a radial flange extending from said first open end of said cylindrical wall, said flange supporting a first and second upstanding tabs extending substantially normally therefrom.

11. The stopcock of claim 10, wherein said first and second tabs are assymetically arrayed about the longitudinal axis of said stopcock body.
